(19) United States
(12) Patent Application Publication
(10) Pub. No.: US 2024/0293795 A1
(43) Pub. Date: Sep. 5, 2024

(54) ZIRCONIUM-BASED METAL ORGANIC FRAMEWORK FOR USING AS A HEAVY METAL ADSORBENT IN CONDENSATE AND PREPARATION METHOD THEREOF

(71) Applicant: PTT EXPLORATION AND PRODUCTION PUBLIC COMPANY LIMITED, Bangkok (TH)

(72) Inventors: Sunatda ARAYACHUKIAT, Bangkok (TH); Taradon PIROMCHART, Bangkok (TH); Kanokwan KONGPATPANICH, Rayong (TH); Vetiga SOMJIT, Rayong (TH); Taweesak PILA, Rayong (TH); Vitsarut TANGSERMVIT, Rayong (TH); Panchanit PIYAKEERATIKUL, Rayong (TH)

(21) Appl. No.: 18/575,760

(22) PCT Filed: Jun. 29, 2022
(86) PCT No.: PCT/IB2022/056061

§ 371 Date: Dec. 29, 2023

(30) Foreign Application Priority Data
Jun. 30, 2021 (TH) 2101003971

(51) Int. Cl. B01J 20/22; B01J 20/28; B01J 20/30

(52) U.S. Cl. CPC B01J 20/226; B01J 20/28061; B01J 20/28064; B01J 20/28069; B01J 20/28083; B01J 20/3071; B01J 20/3078; B01J 20/3085

(57) ABSTRACT

The present invention relates to a zirconium-based metal organic framework comprising at least a tetravalent zirconium ion (Zr⁴⁺) and a bidentate or tridentate linking ligand bonding the said tetravalent zirconium ion (Zr⁴⁺). Moreover, the present invention also relates to a method for preparing the zirconium-based metal organic framework comprising the steps of: (a) preparing a reaction mixture comprising a zirconium compound, a linking ligand and, optionally, a modulating agent in a solvent; (b) heating the reaction mixture obtained from step (a); and (c) washing a reaction product obtained from step (b) with the solvent and drying the reaction product. The zirconium-based metal organic framework according to the present invention is suitable for using in a process for removing heavy metals in the condensate, especially using in the adsorption, removal, or reduction of arsenic and mercury contents in the condensate.

### TECHNICAL FIELD

Chemistry related to a zirconium-based metal organic framework for using as a heavy metal adsorbent in condensate and a preparation method thereof

### BACKGROUND OF THE INVENTION

In the petroleum exploration and production industry, the heavy metal contaminants, for example, arsenic (As) and mercury (Hg) can generally be found in a petroleum product which is a natural hydrocarbon compound obtained from the production process, including crude oil, natural gas, and natural gas liquid (NGL), also called condensate or natural gas condensate. These heavy metal contaminants cause disadvantages in terms of toxicity and corrosiveness which is a problem for performing next step where the petroleum product, especially the condensate, is used as a starting material, for example, in the petrochemical industry. The arsenic and mercury contained in a condensate may be in the form of various compounds, for example, mercury sulfide (HgS), mercury oxide (HgO), Arsenopyrite (AsFeS), etc.

Accordingly, to satisfy the needs to reduce or remove the heavy metal contaminants, especially arsenic and mercury compounds contained in the condensate, there is an attempt to develop methods and materials for using in the adsorption of those contaminants.

A metal-organic framework wherein the structure consists of a metal cluster and an organic linking ligand is deemed as a novel porous material which receives great attention and found in various applications, for example, gas storage, gas separation, chemical sensor, and heterogeneous catalysis, etc. Industrially, the metal-organic framework is another interesting option for using as an adsorbent with the adsorption property as required depending on the structure and porosity that can be adjusted according to the type of metal cluster and linking ligand selected.

Examples of invention related to the development of metal-organic framework for using as a contaminant adsorbent are shown below.

WO 2020/130953 A1 discloses the copper-based metal-organic framework for using in a removal of carbon dioxide (CO₂) and other contaminants, for example, mercury, arsenic and hydrogen sulfide (H₂S) from petroleum. The said copper-based metal-organic framework is obtained by a method comprising mixing copper (II) (Cu(II)) salt and 2,5-dibromobenzene-1,4-dicarboxylic acid, dimethylformamide (DMF) and methanol together, heating such mixture, and collecting the product.

WO 2020/130954 A1 discloses the copper-based metal-organic framework for using in a removal of carbon dioxide (CO₂) and other contaminants such as Hg, As, and hydrogen sulfide (H₂S) from petroleum. The said copper-based metal-organic framework is obtained by a method comprising the steps of mixing copper (II) (Cu(II)) salt and 1,2,4,5-tetrabromobenzene dicarboxylic acid, methanol and water together, heating such mixture, and collecting the product.

U.S. Pat. No. 10,260,148 B2 discloses a porous material including the metal-organic framework and a porous organic polymer for purifying electronic gas and removing mercury from the hydrocarbon stream.

### SUMMARY OF THE INVENTION

The first aspect of the present invention relates to the zirconium-based metal organic framework for using as a heavy metal adsorbent in a condensate comprising at least a tetravalent zirconium ion (Zr⁴⁺) and a bidentate or tridentate linking ligand bonding the said tetravalent zirconium ion (Zr⁴⁺), wherein the zirconium-based metal organic framework according to the present invention may be subject to a surface treatment with a solution of alkali metal hydroxide to improve or enhance the efficiency in the heavy metal adsorption in the condensate.

The second aspect of the present invention relates to a method for preparing the zirconium-based metal organic framework for using as a heavy metal adsorbent in the condensate comprising the steps of:
(a) preparing a reaction mixture comprising a zirconium compound, a linking ligand and, optionally, a modulating agent in a solvent;
(b) heating the reaction mixture obtained from step (a) at a temperature ranging from 80-150° C. for 6-48 hours; and
(c) washing a reaction product obtained from step (b) with the solvent and drying the reaction product at a temperature ranging from 80-150° C. for 6-15 hours.

Optionally, the method for preparing the zirconium-based metal organic framework according to the present invention may further comprise the step of (d) contacting the reaction product obtained from step (c) with an aqueous solution of alkali metal hydroxide at ambient temperature for 12-36 hours.

The third aspect of the present invention relates to a process for removing heavy metals in the condensate comprising contacting the condensate with the adsorbent comprising the zirconium-based metal organic framework according to the present invention.

An objective of the present invention is to provide the zirconium-based metal organic framework with the abilities to adsorb, remove, or reduce the contaminants which are heavy metal compounds, especially arsenic (As) and mercury (Hg) which may be in the form of compounds containing such heavy metals in the condensate.

Another objective of the present invention is to provide the method for preparing the zirconium-based metal organic framework where the framework's properties can be optimized for using as the aforementioned contaminant adsorbent in the condensate.

Moreover, the present invention is also aimed to provide the process for removing the aforementioned contaminants from the condensate by using an adsorbent which is a zirconium-based metal organic framework according to the present invention or an adsorbent comprising a zirconium-based metal organic framework according to the present invention.

The zirconium-based metal organic framework prepared and characterized according to the present invention showed the great efficiency in adsorbing heavy metal compounds, especially arsenic and mercury in the condensate. It can remove up to about 85% of arsenic compound in the condensate and can remove up to about 99% of mercury compound in the condensate. Moreover, it was also found that the zirconium-based metal organic framework according to the present invention gave significantly higher percent removal of arsenic and mercury compounds in the condensate than other types of metal-organic framework commonly available.

### BRIEF DESCRIPTION OF THE DRAWINGS

FIG. 1 is a powder X-ray diffraction pattern of Example 1 (FIG. 1(a)), Example 2 (FIG. 1 (b)), and Example 3 (FIG. 1(c)) which are examples of the zirconium-based metal organic framework according to the present invention.

FIG. 2 is a nitrogen adsorption-desorption isotherm of Example 1 (FIG. 1(a)), Example 2 (FIG. 1(b)), and Example 3 (FIG. 1(c)) which are examples of the zirconium-based metal organic framework according to the present invention.

FIG. 3 is the nitrogen adsorption-desorption isotherm of Example 1 which is the zirconium-based metal organic framework according to the present invention and Comparative Examples A and B.

### DETAILED DESCRIPTION

Any aspects shown herein shall encompass the application to other aspects of the present invention as well, unless stated otherwise.

Technical terms and scientific terms used herein have meanings as understood by a person of ordinary skill in the art, unless specified otherwise.

Throughout the present invention, the term “about” is used to indicate that any values appearing or shown herein may be varied or deviate. Such variation or deviation may be caused by equipment error, or method used to determine the values.

The terms “consist(s) of,” “comprise(s),” “contain(s),” and “include(s)” are open-end verbs. For example, any method which “consists of,” “comprises,” “contains” or “includes” one component or multiple components or one step or multiple steps is not limited to only one component or one step or multiple steps or multiple components, but also encompasses components or steps that are not specified.

Tools, devices, methods, materials, or chemicals mentioned herein, unless specified otherwise, mean the tools, devices, methods, materials, or chemicals generally used or practiced by a person skilled in the art.

All components and/or methods disclosed and claimed in the present invention are intended to cover the aspects of the invention obtained from an action, a practice, a modification or a change of any factors which does not require any experiment that is substantially different from the present invention and gives properties and utility and provides the same effect as the aspects of the present invention according to the judgement of a person of ordinary skill in the art, although not specifically stated in the claims. Accordingly, substitutions or analogues of the aspects of the present invention and any slight modifications or changes that is clearly apparent to a person of ordinary skill in the art, are considered to be within the spirit, scope, and concept of the present invention as well.

The term “condensate” according to the present invention shall encompass the “condensate oil” or “natural gas liquid (NGL)” or “natural gas condensate” generally used in the art. As an example, the term “condensate” encompasses a mixture of liquid hydrocarbon having a molecular weight in a range of hydrocarbon containing from 1-14 carbon atoms, preferably 3-14 atoms.

The aspects of the present invention will now be described in more detail.

### Zirconium-Based Metal Organic Framework

The first aspect of the present invention relates to the zirconium-based metal organic framework for using as a heavy metal adsorbent in a condensate comprising at least a tetravalent zirconium ion (Zr⁴⁺) and a bidentate or tridentate linking ligand bonding the said tetravalent zirconium ion (Zr⁴⁺).

In an alternative embodiment, the said zirconium-based metal organic framework is subject to a surface treatment with a solution of alkali metal hydroxide, especially subject to the surface treatment with the solution of alkali metal hydroxide with a controlled pH in a range of 7-12, preferably in a range of 7-8.

As an example, the surface treatment with such solution of alkali metal hydroxide may be conducted at ambient temperature for 12-36 hours.

The alkali metal hydroxide suitable for the surface treatment according to the present invention may be selected from a group consisting of sodium hydroxide, potassium hydroxide, and a mixture thereof.

Preferably, such alkali metal hydroxide solution is an aqueous solution of sodium hydroxide.

The linking ligand may be selected from a group consisting of 1,4-benzenedicarboxylic acid, 1,3,5-benzenetricarboxylic acid, But-2-enedioic acid, and a mixture thereof.

In a more specific embodiment, the tetravalent zirconium ion (Zr⁴⁺) is derived either from zirconium tetrachloride, zirconium oxychloride, zirconium oxychloride octahydrate, zirconium dioxide, zirconium tetrahydroxide, or a mixture thereof.

Preferably, the tetravalent zirconium ion (Zr⁴⁺) is derived from zirconium tetrachloride or zirconium oxychloride octahydrate.

The zirconium-based metal organic framework according to the present invention comprises a cluster node of 6 zirconium atoms (Zr₆ cluster node) and 8 oxygen atoms partially linked to the linking ligand.

Preferably, the zirconium-based metal organic framework has a mole ratio of the tetravalent zirconium ion (Zr⁴⁺) to the linking ligand in a range of 1:1-3.

Moreover, the zirconium-based metal organic framework has an average BET surface area in a range of 300-1000 m²/g.

Preferably, the zirconium-based metal organic framework according to the present invention has an average pore volume in a range of 0.2-1.2 cm³/g and has an average pore diameter in a range of 3-5 nm.

Also preferably, the zirconium-based metal organic framework has a nitrogen adsorption-desorption isotherm type I or IV.

The zirconium-based metal organic framework according to the present invention is suitable especially for using as an arsenic and/or mercury adsorbent in the condensate.

Moreover, the present invention also relates to an adsorbent comprising the zirconium-based metal organic framework with the aforementioned characteristics according to the present invention.

The second aspect of the present invention relates to the method for preparing the zirconium-based metal organic framework for using as a heavy metal adsorbent in the condensate.

The method for preparing the zirconium-based metal organic framework for using as the heavy metal adsorbent in the condensate according to the present invention comprises the steps of:
(a) preparing a reaction mixture comprising a zirconium compound, a linking ligand and, optionally, a modulating agent in a solvent;
(b) heating the reaction mixture obtained from step (a) at a temperature ranging from 80-150° C. for 6-48 hours; and
(c) washing a reaction product obtained from step (b) with the solvent and drying the reaction product at a temperature ranging from 80-150° C. for 6-15 hours.

The method for preparing the zirconium-based metal organic framework according to the present invention may further comprise the step (d) of contacting a reaction product obtained from step (c) with an aqueous solution of alkali metal hydroxide at ambient temperature for 12-36 hours.

Preferably, in step (d), pH of the aqueous solution of alkali metal hydroxide is controlled in a range of 7-12, preferably in a range of 7-8.

Such alkali metal hydroxide used in step (d) can be selected from a group consisting of sodium hydroxide, potassium hydroxide, and a mixture thereof.

Preferably, the aqueous solution of alkali metal hydroxide according to the method of the present invention is the aqueous solution of sodium hydroxide.

In a further aspect, the method for preparing the said zirconium-based metal organic framework further comprises step (e) of washing a product obtained from step (d) with the solvent and drying the product at a temperature ranging from 80-150° C. for 6-12 hours. Preferably, in step (e), the solvent is water.

In a specific embodiment, a mole ratio of the zirconium compound to the linking ligand in step (a) is in a range of 1:1-3.

Alternatively, a mole ratio of the zirconium compound to the modulating agent in step (a) is in a range of 1:4-6.

Optionally, a mole ratio of the zirconium compound to the modulating agent in step (a) is in a range of 1:300-400.

In a specific embodiment of the present invention, the method for preparing the zirconium-based metal organic framework comprises the steps of:
(a) preparing the reaction mixture comprising the zirconium compound and the linking ligand in the solvent;
(b) heating reaction mixture obtained from step (a) at the temperature ranging from 100-150° C. for 12-36 hours;
(c) washing the reaction product obtained from step (b) with the solvent and drying the reaction product at the temperature ranging from 100-150° C. for 6-15 hours;
(d) contacting the reaction product obtained from step (c) with the aqueous solution of alkali metal hydroxide with the controlled pH in a range of 7-12 at ambient temperature for 12-36 hours; and
(e) washing the product obtained from step (d) with the solvent and drying the product at the temperature ranging from 80-150° C. for 6-12 hours;
wherein in the mole ratio of the zirconium compound to the linking ligand in step (a) is in the range of 1:1-3.

In a more specific embodiment of the present invention, the method for preparing the zirconium-based metal organic framework comprises the steps of:
(a) preparing the reaction mixture comprising the zirconium compound, the linking ligand and the modulating agent in the solvent;
(b) heating the reaction mixture obtained from step (a) at the temperature ranging from 80-150° C. for 24-48 hours;
(c) washing the reaction product obtained from step (b) with the solvent and drying the reaction product at the temperature ranging from 100-150° C. for 6-12 hours;
(d) contacting the reaction product obtained from step (c) with the aqueous solution of alkali metal hydroxide with the controlled pH in the range of 7-12 at ambient temperature for 12-36 hours; and
(e) washing the product obtained from Step (d) with the solvent and drying the product at a temperature ranging from 80-150° C. for 6-12 hours;
wherein the mole ratio of the zirconium compound to the linking ligand in step (a) is in the range of 1:1-3 and the mole ratio of the zirconium compound to the modulating agent in step (a) is in the range of 1:300-400.

In another specific embodiment of the invention, the method for preparing the zirconium-based metal organic framework comprises the steps of:
(a) preparing the reaction mixture comprising the zirconium compound, the linking ligand and the modulating agent in the solvent;
(b) heating the reaction mixture obtained from step (a) at the temperature ranging from 90-110° C. for 4-8 hours;
(c) washing the reaction product obtained from step (b) with the solvent and drying the reaction product at the temperature ranging from 80-150° C. for 6-12 hours,
wherein the mole ratio of the zirconium compound to the linking ligand in step (a) is in the range of 1:1-3 and the mole ratio of the zirconium compound to the modulating agent in step (a) is in the range of 1:4-6.

The preferred zirconium compound according to the method of the present invention may be selected from a group consisting of zirconium tetrachloride, zirconium oxychloride, zirconium oxychloride octahydrate, zirconium dioxide, zirconium tetrahydroxide, and a mixture thereof.

More preferably, the zirconium compound is zirconium oxychloride octahydrate or zirconium tetrachloride.

The preferred linking ligand according to the method of the present invention may be selected from a group consisting of 1,4-benzenedicarboxylic acid, 1,3,5-benzenetricarboxylic acid, But-2-enedioic acid, and a mixture thereof.

The preferred modulating agent according to the method of the present invention may be selected from a group consisting of formic acid, acetic acid, propionic acid, and a mixture thereof.

Even more preferably, the modulating agent is formic acid or acetic acid.

According to the method of the present invention, the solvent usable in steps (a) and (c) may be water and/or the organic solvent, e.g., acetone, dimethylformamide (DMF), dimethyl sulfoxide (DMSO), alcohol (alcohol) such as methanol, ethanol, etc.

Specifically, in step (a), the solvent may be selected from a group consisting of dimethylformamide, water, dimethyl sulfoxide, methanol, ethanol, and a mixture thereof.

Preferably, in step (a), the solvent is dimethylformamide or water.

Specifically, in step (c), the solvent may be selected from a group consisting of dimethylformamide, acetone, methanol, ethanol, water, and a mixture thereof.

The third aspect of the present invention relates to the process for removing heavy metals in the condensate comprising contacting the condensate with the adsorbent comprising the zirconium-based metal organic framework characterized according to the present invention or prepared according to the method of the present invention.

In a specific embodiment, the heavy metal removal process according to the present invention comprises contacting the condensate with the adsorbent which is performed at a temperature ranging from 18-80° C. and a pressure ranging from 1-30 bars.

### EXAMPLE

Now, the invention will be described in more detail with reference to the examples of experiment and the accompanying drawings. However, these examples shall not be deemed to limit the scope of the present invention.

### 1. Preparation of Examples of Zirconium-Based Metal Organic Framework

Prepare the examples of zirconium-based metal organic framework according to the present invention (Examples 1-3) using the chemicals, methods, and conditions as follows.

### Example 1

Dissolved ZrOCl₂·8H₂O and 1,4-benzenedicarboxylic acid (the mole ratio of ZrOCl₂·8H₂O to 1,4-benzenedicarboxylic acid equal to about 1:1) in DMF. The resulting mixed solution (that is, the reaction mixture) was subject to sonication for 1 minute. Then, reacted by heating such mixed solution in an oven at a temperature of 120° C. for 24 hours. After completion, the resulting solid product was collected by centrifuging. The product was washed with DMF (3 times) and acetone (3 times), and then dried at a temperature of 150° C. under vacuum for 12 hours. Then, the dried product surface was treated by stirring in an aqueous solution of NaOH for 24 hours. After completion, washed with deionized water (DI) (3 times) and dried at a temperature of 150° C. under vacuum for 12 hours to obtain the final product as a white solid.

### Example 2

Dissolved ZrOCl₂·8H₂O and But-2-enedioic acid (the mole ratio of ZrOCl₂·8H₂O to But-2-enedioic acid equal to about 1:1) in a mixture between water and formic acid (the mole ratio of ZrOCl₂·8H₂O to formic acid equal to about 1:6). The resulting mixed solution (that is, the reaction mixture) was subject to sonication for 5 minutes. Then, reacted by heating such mixed solution in an oven at a temperature of 100° C. for 6 hours. After completion, the resulting solid product was collected by centrifuging. The product was washed with deionized water (3 times) and ethanol (3 times), and then dried at a temperature of 150° C. under vacuum for 12 hours to obtain the final product as a white solid.

### Example 3

Dissolved ZrCl₄ and 1,3,5-benzenetricarboxylic acid (the mole ratio of ZrCl₄ to 1,3,5-benzenetricarboxylic acid at about 1:3) in a mixture between DMF and formic acid (the mole ratio of ZrCl₄ to formic acid at about 1:358). The mixed solution was subject to sonication for 20 minutes. Then, reacted by heating such mixed solution in an oven at a temperature of 130° C. for 48 hours. After completion, the resulting solid product was collected by centrifuging. The product was washed with DMF (3 times) and ethanol (3 times), and then dried at a temperature of 150° C. under vacuum for 12 hours. Then, such dried product surface was treated by stirring in an aqueous solution of NaOH for 24 hours. After completion, washed with DI water (3 times) and dried at a temperature of 120° C. under vacuum for 12 hours to obtain the final product as a white solid.

### 2. Characterization of Examples of Zirconium-Based Metal Organic Framework

The above prepared examples of zirconium-based metal organic framework with different types of linking ligands (Examples 1-3) were further characterized using X-ray powder diffraction technique (XRD) to confirm the structure of the synthesized zirconium-based metal organic framework and nitrogen adsorption measurement technique (N₂ adsorption) to characterize the porosity, the average BET surface area, and the pore volume of such examples.

### 2.1. Analysis Results by X-Ray Powder Diffraction Technique

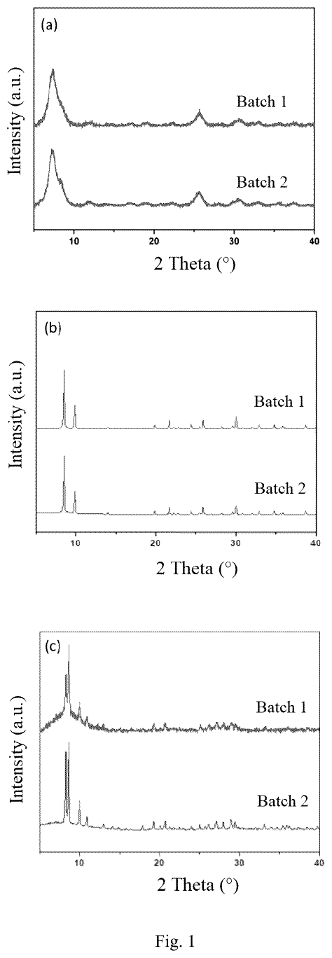

FIG. 1(a) shows the XRD pattern of Example 1 obtained from the preparation of two separate batches which is in the form of a gel of the product consisting of nanoparticles of the material. The gel form is one of the important characteristics different from the metal organic framework commonly available which is usually synthesized as a microparticle crystal or powder. From FIG. 1(a), the gel form of the material can be observed from the broaden peak.

The gel form of the material is one of the advantages as it is a vicious liquid with good stability. The material thus can be used conveniently compared to a powdered material which needs to be formed, for example, into granules before use.

Moreover, a small peak was also found at 2 theta of 6.5° which was a diffraction peak from the defect site in the structure of the metal organic framework of Example 1. The peak at this position would not be found in the simulated pattern of ideal UiO-66 crystal.

FIGS. 1(b) and 1(c) show the XRD pattern of Examples 2 and 3 which were obtained from the preparation of two separate batches, respectively. It can be observed that in Example 3, 3 additional peaks from the defect site in the structure were found at 2 theta of about 7°, indicating that there were a lot of structural defects at Zr node of Example 3.

Moreover, from the XRD pattern of the three examples, the peaks were found at 2 theta of about 7°-9°, corresponding to the diffraction plane (111) and (200) of the 6-zirconium atom cluster (Zr₆ cluster). This shows that the tetravalent zirconium ions (Zr⁴⁺) in the structure of Examples 1, 2 and 3 were in the form of 6-zirconium atom cluster.

### 2.2. Analysis Results by Nitrogen Adsorption Technique

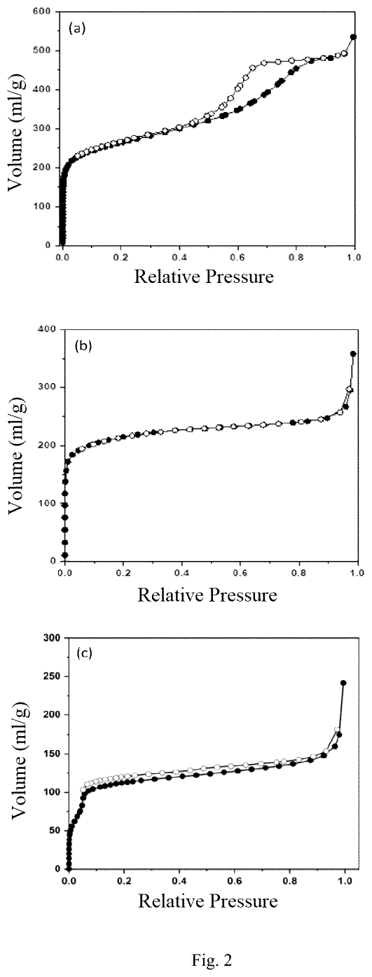

FIG. 2(a) shows the nitrogen adsorption-desorption isotherm type IV of Example 1 which was evaluated as a microporous material with mesopores caused by the gel form of the material. The nitrogen adsorption-desorption isotherm type IV characteristic has a positive effect on the adsorption of arsenic compounds, particularly arsenate (As(V), usually in the form of oxide compound, such as a large H₃AsO₄ which requires a larger area inside the adsorbent.

FIGS. 2(b) and 2(c) show the nitrogen adsorption-desorption isotherm type I of Examples 2 and 3, respectively. Example 2 shows lower gas adsorption compared to Example 1 as a result of the smaller size of linking ligand in the structure of Example 2.

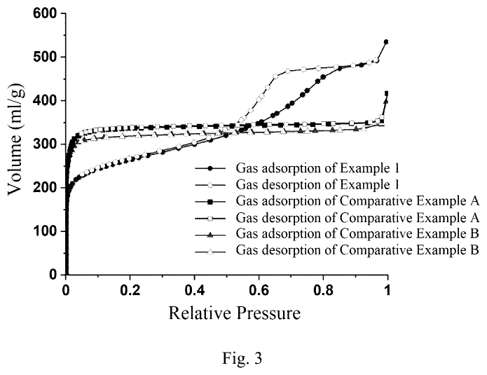

FIG. 3 shows the nitrogen adsorption-desorption isotherm of Example 1 that is different from comparative examples A and B which are zirconium-based metal organic frameworks and have the same type of linking ligands. The comparative examples are as described in detail below.

The analysis results of the average BET surface area and the pore volume of Examples 1-3 and the comparative examples are as shown in Table 1, provided that Comparative Examples A and B are as follows:
1 Comparative Example A is a commercially available zirconium-based metal organic framework (UiO-66); and
2. Comparative Example B is a commercially available zirconium-based metal organic framework (UiO-66) subject to surface treatment with an aqueous solution of sodium hydroxide with the controlled pH in a range of 7-12 for 24 hours.

TABLE 1

| | Average BET surface area | Pore volume |
|---|---|---|
| Example | (m²/g) | (cm³/g) |
| Comparative A | 1335 | 0.60 |
| Comparative B | 1335 | 0.59 |
| 1 | 974 | 0.81 |
| 2 | 800 | 0.55 |
| 3 | 400 | 0.34 |

### 3. Study on the Effect of pH of the Aqueous Solution of Sodium Hydroxide

According to the present invention, the efficiency in adsorbing the compounds of arsenic (As) and mercury (Hg) has been improved or enhanced by treating the surface of the material with the aqueous solution of alkali metal hydroxide, for example, sodium hydroxide, to increase the amount of hydroxy group (—OH) on the surface of the zirconium-based metal organic framework according to the present invention. Such increased amount of hydroxy group will increase the arsenic-specific active site due to the oxophilicity of arsenic which tends to form bonds with oxygen atoms.

The experiment was conducted to further study the effect of pH of the aqueous solution of sodium hydroxide used in the surface treatment step for the zirconium-based metal organic framework according to the method of the present invention by comparing the efficiencies in adsorbing arsenic compounds of the zirconium-based metal organic framework examples obtained by using the aqueous solution of sodium hydroxide with different pH, i.e., pH of 7, 8, 9, and 10, in the surface treatment step according to the method of the present invention. In the experiment, the pH of the aqueous solution of sodium hydroxide before being added to the dried zirconium-based metal organic framework example in step (c) (herein represented by pH_{NaOH aq} before treatment) and the pH of parts of the aqueous solution of sodium hydroxide during surface treatment (herein represented by pH_{NaOH aq} during treatment) were measured. The surface treatment is conducted at ambient temperature for 24 hours.

The initial removal of arsenic compounds in water was tested using the zirconium-based metal organic framework examples obtained by using the aqueous solution of sodium hydroxide at the different pH. The details are as follows.

### Test Method

1. The test zirconium-based metal organic framework example according to the present invention was activated by heating at a temperature of 150° C. under vacuum for 24 hours.
2. 2 mg of activated metal organic framework was added to a 20 ml flask.
3. 10 ml of aqueous solution containing either As(III) or As(V) was added to such flask and left for 1 hour.
4. The zirconium-based metal organic framework example was extracted by centrifuging at 12,000 rpm for 5 minutes.
5. The concentration of the arsenic compounds remaining in the aqueous solution was measured using the graphite furnace atomic absorption spectrometry technique (GFAAS), and the concentration of the mercury compound remaining in the aqueous solution was measured using the mercury analyzer (Hg Analyzer).
6. The percent removal of arsenic compounds was calculated by comparing the amount of As(III) or As(V) before and after adsorption with the zirconium-based metal organic framework examples and the arsenic compound adsorption ability which is the amount (in mg) of adsorbed arsenic compound versus the amount (in g) of used adsorbent was determined.

The experiment result is shown in Table 2.

TABLE 2

| | | As(III) | | As(V) | |
|---|---|---|---|---|---|
| pH_{NaOH aq} | pH_{NaOH aq} | Adsorption | % | Adsorption | % |
| before | during | ability | Re- | ability | Re- |
| treatment | treatment | (mg/g) | moval | (mg/g) | moval |
| 7 | 7 | 52.12 | 26.44 | 101.93 | 45.74 |
| 8 | 8 | 46.83 | 21.85 | 102.91 | 46.18 |
| 9 | 9 | 8.76 | 4.44 | 98.21 | 44.07 |
| 10 | 10 | 15.96 | 8.42 | 83.79 | 39.10 |

The experiment result in Table 2 shows that the zirconium-based metal organic framework examples prepared by treating with the aqueous solution of sodium hydroxide with pH of 7-10 had a good removal ability for the arsenic compounds, i.e., As(III) and As(V). The zirconium-based metal organic framework examples prepared by treating with the aqueous solution of sodium hydroxide with pH of 7 and 8 had the highest removal ability for arsenic.

Moreover, the initial adsorption efficiencies for the arsenic compounds, i.e., As(III) and As(V), in water of the zirconium-based metal organic framework examples according to the present invention and the comparative examples were compared according to the above test method. The experiment result is as shown in Table 3 below.

### Description of the Metal Organic Framework Examples Used in the Experiment

1. Example 1 is a zirconium-based metal organic framework having 1,4-benzene #dicarboxylic acid as a ligand prepared according to the method of the present invention.
2. Example 2 is a zirconium-based metal organic framework having But-2-enedioic acid as a ligand prepared according to the method of the present invention.
3. Comparative Example A is a zirconium-based metal organic framework having 1,4-benzenedicarboxylic acid as a commercially available linking ligand (UiO-66).
4. Comparative Example B is a zirconium-based metal organic framework having 1,4-benzenedicarboxylic acid as a commercially available linking ligand (UiO-66) that is subject to further surface treatment with the aqueous solution of sodium hydroxide with the controlled pH in a range of 7-12 for 24 hours.
5. Comparative Example C is a zirconium-based metal organic framework having biphenyl-4,4′-dicarboxylic acid as a commercially available linking ligand (UiO-67)

TABLE 3

| | As(III) | | As(V) | |
|---|---|---|---|---|
| | Adsorption | % | Adsorption | % |
| Example | ability (mg/g) | Removal | ability (mg/g) | Removal |
| Comparative A | 42.54 | 16.44 | 64.62 | 25.10 |
| Comparative B | 78.23 | 41.72 | 69.15 | 28.27 |
| Comparative C | 35.34 | 14.38 | 69.15 | 28.27 |
| (UiO-67) | | | | |
| 1 | 109.20 | 54.60 | 87.66 | 55.10 |
| 2 | 84.59 | 52.87 | 45.96 | 39.40 |

From the experiment result, when comparing between the structures with the same type of metal centres and linking ligands, it was found that surface treatment with the aqueous solution of sodium hydroxide with pH of 7-12 (Examples 1-3 and Comparative Example B) can significantly improve or enhance the ability to adsorb arsenic compounds, particularly As(III), of the zirconium-based metal organic framework, compared to the example that is not subject to surface treatment (Comparative Example A). However, Examples 1 and 2 prepared using chemicals, ratios, and particular steps according to the method of the present invention gave the higher percent removal of arsenic compounds, where Example 1 gave the highest percent removal of As(III) and As(V).

### 4. Test on Adsorption Efficiency for Arsenic and Mercury Compounds in the Condensate

The efficiency in adsorbing arsenic and mercury compounds in the condensate examples obtained from two different sources was tested using the adsorbent, i.e., the zirconium-based metal organic framework examples prepared according to the method of the present invention (Examples 1-3) and Comparative Examples A-G which are the metal organic frameworks containing the different types of metal centres and linking ligands. The details are as follows.

### Description of the Metal Organic Framework Examples Used in the Experiment

1. Example 1 is a zirconium-based metal organic framework having 1,4-benzene dicarboxylic acid as a ligand prepared according to the method of the present invention.
2. Example 2 is a zirconium-based metal organic framework having But-2-enedioic acid as a ligand prepared according to the method of the present invention.
3. Example 3 is a zirconium-based metal organic framework having 1,3,5-benzene tricarboxylic acid as a ligand prepared according to the method of the present invention.
4. Comparative Example A is a zirconium-based metal organic framework having 1,4-benzenedicarboxylic acid as a commercially available linking ligand (UiO-66).
5. Comparative Example C is a zirconium-based metal organic framework having biphenyl-4,4′-dicarboxylic acid as a commercially available linking ligand (UiO-67).
6. Comparative Example D is a manganese-based metal organic framework (Mn-MOF) having 2,5-dioxido-1,4-benzenedicarboxylate as a linking ligand.
7. Comparative Example E is an iron-based metal organic framework having 1,3,5-benzenetricarboxylic acid as a linking ligand that is subject to surface treatment with the aqueous solution of sodium hydroxide with the controlled pH in a range of 8-12 for 24 hours.

### Test Method

1. The test the metal organic framework examples (Examples 1-3 and Comparative Example aforementioned) were activated by heating at a temperature of 150° C. under vacuum for 24 hours.
2. 50 mg of activated metal organic framework example was added to a 100 ml flask.
3. 37 ml of condensate was added to such flask and left for 1 hour.
4. The zirconium-based metal organic framework example was extracted by centrifuging at 12,000 rpm for 5 minutes.
5. The concentration of arsenic compound remaining in the condensate was measured using the graphite furnace atomic absorption spectrometry technique (GFAAS) and the concentration of mercury compound remaining in aqueous solution was measured using the mercury analyzer (Hg Analyzer).
6. The arsenic and mercury compound adsorption abilities were calculated from the amount (in mg) of adsorbed arsenic or mercury compound versus the amount (in g) of used adsorbent.
7. The percent removal of arsenic and mercury compounds was calculated by comparing the amount of arsenic and mercury compounds before and after adsorption with the zirconium-based metal organic framework examples.

### Test Result

The test result on the efficiency in adsorbing arsenic and mercury compounds in the condensate examples derived from Sources 1 and 2 using the different types of metal organic frameworks (Examples 1-3 and Comparative Examples aforementioned) as the adsorbent is shown in Table 4.

TABLE 4

| | | Arsenic | % | Mercury | % |
|---|---|---|---|---|---|
| | | compound | Removal | compound | Removal |
| | | adsorption | of | adsorption | of |
| Conden- | | ability | arsenic | ability | mercury |
| sate | Adsorbent | (mg/g) | compound | (mg/g) | compound |
| Source 1 | Comparative | 6.30 | 52.10 | 3.01 | 96.00 |
| | Example A | | | | |
| | Comparative | 5.80 | 48.40 | 3.11 | 99.20 |
| | Example C | | | | |
| | Example 1 | 18.70 | 85.19 | 1.42 | 99.35 |
| | Example 2 | 15.93 | 73.73 | 1.40 | 99.89 |
| | Example 3 | 15.44 | 70.88 | 1.41 | 99.61 |
| Source 2 | Comparative | 3.40 | 54.30 | 11.31 | 96.00 |
| | Example A | | | | |
| | Comparative | 2.30 | 36.10 | 11.63 | 98.70 |
| | Example C | | | | |
| | Comparative | 2.80 | 43.60 | 11.73 | 99.50 |
| | Example D | | | | |
| | Comparative | 2.90 | 45.30 | 11.74 | 99.60 |
| | Example E | | | | |
| | Example 1 | 10.66 | 71.72 | 5.55 | 82.70 |
| | Example 2 | 9.01 | 61.12 | 5.59 | 83.94 |
| | Example 3 | 9.95 | 67.09 | 4.89 | 72.98 |

From the above experiment result, it was found that when comparing between the zirconium-based metal organic frameworks (i.e., Examples 1-3 and Comparative Examples A, C), the zirconium-based metal organic frameworks according to the present invention had the significantly superior efficiency in adsorbing the arsenic compounds in the condensate (from both Sources 1 and 2) than the comparative examples. That is, when contemplating the structures with the same type of inking ligands, it was found that Example 1 gave the percent removal of arsenic compounds in the condensate from Source 1 of up to about 85% and in the condensate from Source 2 of up to about 71%, while Comparative Example A gave the percent removal of arsenic compounds in the condensate from Source 1 of about 52% and in the condensate from Source 2 of about 54%. From such result, it clearly shows that the method for preparing the zirconium-based metal organic framework according to the method of the present invention can significantly improve the arsenic compound adsorption efficiency.

When contemplating the structures with different types of linking ligands, it was found that Comparative Example C gave the percent removal of arsenic compounds in the condensate from Source 1 of about 48% and from Source 2 of about 36%, while Examples 1-3 gave the percent removal of arsenic compounds in the condensate from Source 1 of about 85% (Example 1), 73% (Example 2), and 71% (Example 3) and in a condensate from Source 2 of about 72% (Example 1), 61% (Example 2), and 67% (Example 3).

Moreover, when comparing between the meta-organic frameworks with different types of metal centres and/or linking ligands (i.e., Examples 1-3 and Comparative Examples D, E), it was found that the zirconium-based metal organic frameworks according to the present invention (Examples 1-3) gave the significantly higher percent removal of arsenic compounds in the condensate from both Sources 1 and 2 than that of the comparative examples.

For example, when contemplating the structures having the same type of linking ligands but having different types of metal centres, it was found that Example 3 (metal centre being zirconium) gave the percent removal of arsenic compounds in the condensate from Source 2 of about 67%, while Comparative Example E (metal centre being iron) gave the percent removal of arsenic compounds in the condensate from Source 2 of about 45%. From such result, it clearly shows that the metal organic frameworks with zirconium metal centre according to the present invention had the significantly superior arsenic compound adsorption efficiency compared to that of the metal organic frameworks with other types of metal centres.

### BEST MODE OF THE INVENTION

Best mode of the invention is as described in the detailed description of the invention.

1. A zirconium-based metal organic framework for using as a heavy metal adsorbent in a condensate comprising at least a tetravalent zirconium ion (Zr⁴⁺) and a bidentate or tridentate linking ligand bonding the said tetravalent zirconium ion (Zr⁴⁺).

2. The zirconium-based metal organic framework of claim 1 which is subject to a surface treatment with a solution of alkali metal hydroxide.

3. The zirconium-based metal organic framework of claim 2, wherein a pH of the solution of alkali metal hydroxide is controlled in a range of 7-12.

4. The zirconium-based metal organic framework of claim 2, wherein the surface treatment with the solution of alkali metal hydroxide is conducted at ambient temperature for 12-36 hours.

5. The zirconium-based metal organic framework of claim 2, wherein the alkali metal hydroxide is selected from a group consisting of sodium hydroxide, potassium hydroxide, and a mixture thereof.

6. The zirconium-based metal organic framework of claim 1, wherein the linking ligand is selected from a group consisting of 1,4-benzenedicarboxylic acid, 1,3,5-benzenetricarboxylic acid, But-2-enedioic acid, and a mixture thereof.

7. The zirconium-based metal organic framework of claim 1, wherein the tetravalent zirconium ion (Zr⁴⁺) is derived either from zirconium tetrachloride, zirconium oxychloride, zirconium oxychloride octahydrate, zirconium dioxide, zirconium tetrahydroxide, or a mixture thereof.

8. The zirconium-based metal organic framework of claim 1, comprising a cluster node of 6 zirconium atoms (Zr₆ cluster node) and 8 oxygen atoms partially linked to the linking ligand.

9. The zirconium-based metal organic framework of claim 1, having a mole ratio of the tetravalent zirconium ion (Zr⁴⁺) to the linking ligand in a range of 1:1-3.

10. The zirconium-based metal organic framework of claim 1, having an average BET surface area in a range of 300-1000 m²/g.

11. The zirconium-based metal organic framework of claim 1, having an average pore volume in a range of 0.2-1.2 cm³/g.

12. The zirconium-based metal organic framework of claim 1, having an average pore diameter in a range of 3-5 nm.

13. The zirconium-based metal organic framework of claim 1, having a nitrogen adsorption-desorption isotherm type I or IV.

14. The zirconium-based metal organic framework of claim 1, for using as an arsenic adsorbent in the condensate.

15. The zirconium-based metal organic framework of claim 1, for using as a mercury adsorbent in the condensate.

16. An adsorbent comprising the zirconium-based metal organic framework of claim 1.

17. A method for preparing a zirconium-based metal organic framework for using as a heavy metal adsorbent in a condensate, the method comprising:
(a) preparing a reaction mixture comprising a zirconium compound, a linking ligand and, a modulating agent in a solvent;
(b) heating the reaction mixture obtained from step (a) at a temperature ranging from 80-150° C. for 6-48 hours; and
(c) washing a reaction product obtained from step (b) with the solvent and drying the reaction product at a temperature ranging from 80-150° C. for 6-15 hours.

18. The method of claim 17 further comprising step (d) of contacting a reaction product obtained from step (c) with an aqueous solution of alkali metal hydroxide at ambient temperature for 12-36 hours.

19. The method of claim 18 wherein in step (d), pH of the aqueous solution of alkali metal hydroxide is controlled in a range of 7-12.

20. The method of claim 18 wherein in step (d), the alkali metal hydroxide is selected from a group consisting of sodium hydroxide, potassium hydroxide, and a mixture thereof.

21. The method of claim 18 further comprising step (e) of washing a product obtained from step (d) with the solvent and drying the product at a temperature ranging from 80-150° C. for 6-12 hours.

22. The method of claim 21, wherein in step (e), the solvent is water.

23. The method of claim 17, wherein a mole ratio of the zirconium compound to the linking ligand in step (a) is in a range of 1:1-3.

24. The method of claim 17, wherein a mole ratio of the zirconium compound to the modulating agent in step (a) is in a range of 1:4-6.

25. The method of claim 17, wherein in a mole ratio of the zirconium compound to the modulating agent in step (a) is in a range of 1:300-400.

26. The method of claim 17,
wherein the mole ratio of the zirconium compound to the linking ligand in step (a) is in the range of 1:1-3.

27. The method of claim 17,
wherein the mole ratio of the zirconium compound to the linking ligand in step (a) is in the range of 1:1-3 and the mole ratio of the zirconium compound to the modulating agent in step (a) is in the range of 1:300-400.

28. The method of claim 17,
wherein the mole ratio of the zirconium compound to the linking ligand in step (a) is in the range of 1:1-3 and the mole ratio of the zirconium compound to the modulating agent in step (a) is in the range of 1:4-6.

29. The method of claim 17, wherein the zirconium compound is selected from a group consisting of zirconium tetrachloride, zirconium oxychloride, zirconium oxychloride octahydrate, zirconium dioxide, zirconium tetrahydroxide, and a mixture thereof.

30. The method of claim 17, wherein the linking ligand is selected from a group consisting of 1,4-benzenedicarboxylic acid, 1,3,5-benzenetricarboxylic acid, But-2-enedioic acid, and a mixture thereof.

31. The method of claim 17, wherein the modulating agent is selected from a group consisting of formic acid, acetic acid, propionic acid, and a mixture thereof.

32. The method of claim 17, wherein in step (a), the solvent is selected from a group consisting of dimethylformamide, water, dimethyl sulfoxide (DMSO), methanol, ethanol, and a mixture thereof.

33. The method of claim 17, wherein in step (c), the solvent is selected from a group consisting of dimethylformamide, acetone, methanol, ethanol, water, and a mixture thereof.

34. A process for removing heavy metals in a condensate comprising contacting the condensate with an adsorbent comprising the zirconium-based metal organic framework of claim 1.

35. The process for removing heavy metals of claim 34, wherein contacting the condensate with the adsorbent is performed at a temperature ranging from 18-80° C. and a pressure ranging from 1-30 bars.
